(19) United States
(12) Patent Application Publication
(10) Pub. No.: US 2023/0310672 A1
(43) Pub. Date: Oct. 5, 2023

(54) Apparatus and Method for Sanitizing an Area

(71) Applicant: Sunnie Pham, Miami, FL (US)

(72) Inventor: Sunnie Pham, Miami, FL (US)

(21) Appl. No.: 17/712,252

(22) Filed: Apr. 4, 2022

(51) Int. Cl. A61L 2/10; G05D 1/00; G05D 1/02

(52) U.S. Cl. CPC A61L 2/10; G05D 1/0094; G05D 1/0246; A61L 2202/11; A61L 2202/14; A61L 2202/16

(57) ABSTRACT

The invention relates to an autonomous sanitation machine comprising a robot assembly and docking station. The robot assembly includes at least one mobility device, at least one optical device, a Comp, and at least one bulb. The at least one optical device is configured to capture data of a space. The Comp is configured to process the captured data and model it as an imprint. The user assigns a sector within said imprint and the at least one bulb is configured to sanitize the user assigned sector. The invention further relates to a method of disinfecting an area using an autonomous sanitation machine.

### TECHNICAL FIELD

The present description relates to autonomous machines, and more specifically to autonomous machines that are configured to sanitize and sterilize a user defined area.

### DESCRIPTION OF RELATED ART

The Covid-19 pandemic brought health and hygiene to the forefront. Using liquid based sanitary products is expensive, time intensive, and leaves a lot of room for human error. Therefore, a market need emerged that desired a low cost and efficient solution to sanitize an area of potential harmful bacteria and viruses wherein the sanitation is carried out in a manner to reduce or eliminate the potential for human error. Duke University has introduced a programmable and timer-based sanitation device called Tru-D® sanitizing systems in their hospitals. See “UV Light Helps Duke Hospitals Fight Transmission of Superbugs” Debbe Geiger, Updated Apr. 28, 2020, Duke Health Blog. https://www.dukehealth.org/blog/uv-light-helps-duke-hospitals-fight-transmission-of-superbugs. Last viewed Sep. 1, 2021. See also, Tru-D hompage. https://tru-d.com/.

The sanitation devices that are currently available on the market are similar to Tru-D®. In particular, the sanitation devices are intended for commercial use—they are bulky, use a type of ultraviolet light that is harmful to humans, lack a means to shut off the harmful UV rays that may be harmful to bystanders, and they must be physically wheeled between different rooms. Additionally, special equipment is needed to handle these devices such as special gloves.

Sanitation is important to hospitals, however there is a growing need to sanitize residential settings as well. Due to the exorbitant costs of medical care in the United States, it is now commonplace for US families to care for sick and elderly family members in the residential family home. Family homes are now outfitted with professional-grade medical equipment and the need to keep the space sanitized is essential. Therefore, a need exists to have a sanitation device capable of delivering the same results as a commercial sanitation device to a residential setting.

### SUMMARY

A first aspect of the present disclosure relates to an autonomous sanitation machine comprising a robot assembly and docking station. The robot assembly includes at least one mobility device, at least one optical device, a Comp, and at least one bulb. The at least one optical device is configured to capture data from a space. The Comp is configured to process the captured data and model it as an imprint. The user assigns a sector within said imprint. The at least one bulb is configured to sanitize the user assigned sector.

In at least one aspect the autonomous sanitation machine, the at least one optical device is a 3D camera.

In at least one aspect the autonomous sanitation machine, the at least one bulb is configured to emit UV-C light.

In at least one aspect the autonomous sanitation machine, the robot assembly is configured to move between a first extended position and a second closed position.

In at least one aspect the autonomous sanitation machine, the at least one bulb is exposed when the robot assembly is in the first extended position and the at least one bulb is inside of the robot assembly when the robot assembly is in the second closed position.

In at least one aspect the autonomous sanitation machine, the robot assembly includes at least one reflector and at least one reflector mount.

In at least one aspect the autonomous sanitation machine, the at least one reflector is substantially curved.

In at least one aspect the autonomous sanitation machine, the at least one reflector is attached to the at least one reflector mount. The at least one reflector is configured to rotate relative to the at least one reflector mount.

Another aspect of the present disclosure relates to a method for sanitizing an area with an autonomous sanitation machine comprising using an autonomous sanitation machine to model a space, defining sectors in the space by a user, selecting the sector to be sanitized, launching a sanitization program causing the autonomous sanitation machine to go to the selected sector, and disinfecting the selected sector.

In at least one aspect of the method for sanitizing an area with an autonomous sanitation machine includes searching for an object by the autonomous sanitation machine, detecting an object, determining if the object is the sector, and terminating the sanitization program if the object is not the sector.

In at least one aspect of the method for sanitizing an area with an autonomous sanitation machine includes alerting the user of the terminated program if the sanitization program is terminated.

In at least one aspect of the method for sanitizing an area with an autonomous sanitation machine included stopping the at least one mobility device of the autonomous sanitation machine when the object is detected.

In at least one aspect of the method for sanitizing an area with an autonomous sanitation machine, includes turning off the at least one bulb of the autonomous sanitation machine when the object is detected.

The above summary is not intended to describe each and every implementation of the concept. In particular, selected features of any illustrative embodiment within this disclosure may be incorporated into additional embodiments unless clearly stated to the contrary.

### BRIEF DESCRIPTION OF THE DRAWINGS

The disclosure may be more completely understood in consideration of the following detailed description of aspects of the disclosure in connection with the accompanying drawings, in which:

FIG. 1 is a perspective view of an autonomous sanitation machine including a robot assembly in a retracted position and a docking station;

FIG. 2 is a top view of the docking station;

FIG. 3 is a bottom view showing the robot assembly disengaged from the docking station;

FIG. 4 is a front view of the robot assembly in an extended position;

FIG. 5 is a right-side view of the robot assembly in the extended position;

FIG. 6 is a cross-section cut along the plane B-B of the robot assembly of FIG. 4;

FIG. 7 is a cross-section cut along the plane C-C of the robot assembly of FIG. 5;

FIG. 8A is a circuit diagram for the docking assembly;

FIG. 8B is a circuit diagram for the robot assembly; and

FIG. 9 is a flowchart of a method for disinfecting an area with an autonomous sanitation machine.

While aspects of the disclosure are amenable to various modifications and alternative forms, specifics thereof have been shown by way of example in the drawings and will be described in detail. It should be understood, however, that the intention is not to limit aspects of the disclosure to the particular embodiment described. On the contrary, the intention of this disclosure is to cover all modifications, equivalents, and alternatives falling within the scope of the disclosure.

### DETAILED DESCRIPTION

As used in this disclosure and the appended claims, the singular forms “a”, “an”, and “the” include plural referents unless the content clearly dictates otherwise. As used in this disclosure and the appended claims, the term “or” is generally employed in its sense including “and/or” unless the content clearly dictates otherwise.

The following detailed description should be read with reference to the drawings. The detailed description and the drawings, which are not necessarily to scale, depict illustrative aspects and are not intended to limit the scope of the invention. The illustrative aspects depicted are intended only as exemplary.

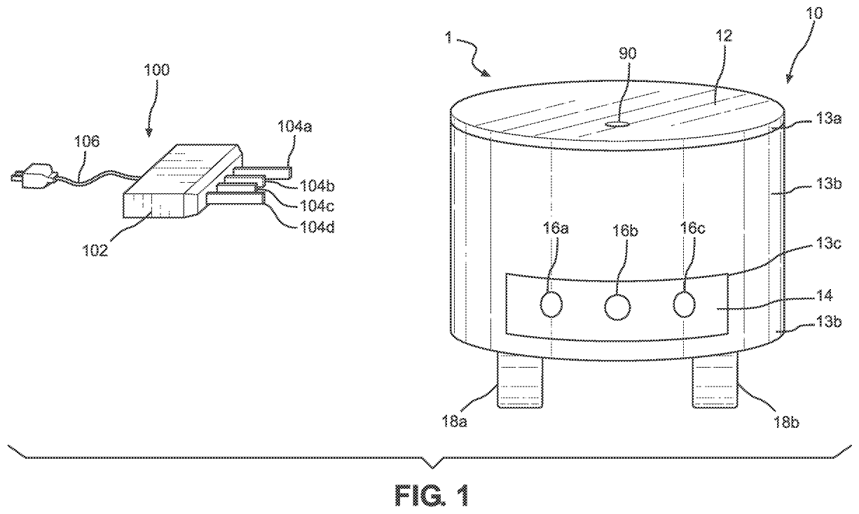

Aspects of the disclosure relate to the autonomous sanitation of an area using a robot assembly and docking station as shown in FIGS. 1 to 9.

FIG. 1 is a perspective view of an autonomous sanitation machine 1 including a robot assembly 10 in a retracted position and docking station 100. The robot assembly 10 comprises a housing 13. In an exemplary embodiment, the housing is in a cylindrical shape, however the housing 13 may be any suitable shape. The cover 12 is secured to a first end 13a of the housing 13. The cover 12 is configured to fit snugly with and detach from the first end 13a of the housing 13, wherein the first end 13a being orientated as the top of the housing 13. The housing 13 and the cover 12 may be aluminum or any suitable material that does not block the transmission of Wi-Fi, Bluetooth, or other wireless communication radio frequencies. The housing 13 includes a window 13c. The window 13c is an opening in the housing 13 and is configured to engage with an optical panel 14.

The robot assembly 10 includes the optical panel 14 that is configured to align with the window 13c and be removably attached to the housing 13.

The robot assembly 10 includes at least one optical device 16a-16c. The at least one optical device 16a-16c is configured to securely attach to optical panel 14. Each optical device 16a-16c may be any combination of LiDAR device, camera, an IR sensor, ultrasonic sensor or any other suitable means for capturing distance, dimensions, or other identifying characters of a given space. The optical panel 14 may be aluminum, a polymer, or any other suitable material that will not interfere with the transmission of radio frequencies.

The cover 12 includes an indicator 90. In the exemplary embodiment, the indicator is a LED light that is capable of emitting multiple colors. In operation, the indicator 90 may emit a green light when the robot assembly 10 is operating, a yellow light when there is a warning or a red light when there is a problem. The indicator 90 may be configured to be continuously on or the indicator 90 may be configured to turn off and on to emit a blinking pattern. It is further contemplated that the indicator 90 may be an audible alarm or a screen. It is further contemplated that the screen may be a touch screen.

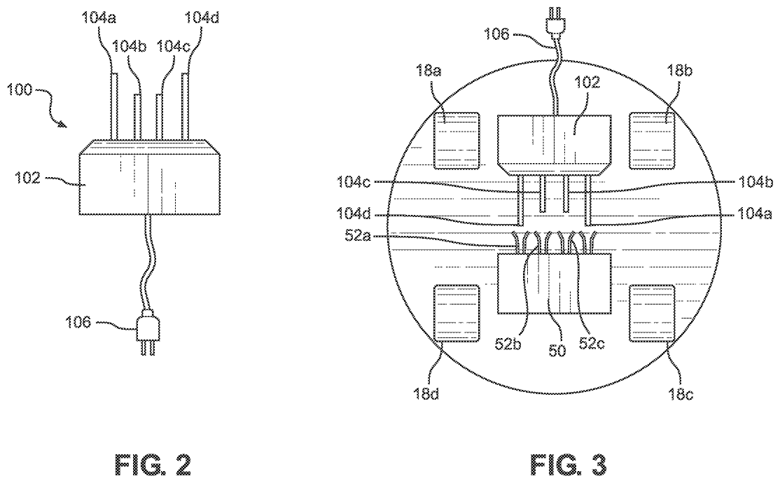

Turning to FIG. 2, the docking station 100 comprises a plug 106 that is configured to insert into a power outlet to receive power from the outlet and convert it into a power supply that is capable of charging the robot assembly 10. The docking station 100 further comprises a casing 102 and at least one connector pin 104a-104d.

Turning to FIG. 3, which is a bottom view of a second end 13b of the robot assembly 10 and docking station 100. Each connector pin 104a-104d is configured to engage with at least one connector prong 52a-52d located on the second end 13b of the housing 13 of the robot assembly 10. The second end 13b being orientated as the bottom of the housing 13. The exemplary embodiment includes four connector pins 104a-104d, however any suitable number of pins may be used. The connector pins 104a-104d may be formed of gold, copper, or any other conductive material.

The robot assembly 10 includes at least one mobility device 18a-18d fixedly attached to the second end 13b of the housing 13 of the robot assembly 10. Each of the at least one mobility device 18a-18d may extend from the housing 13 or be partially disposed therein. In the exemplary embodiment, each of the mobility devices 18a-18d implemented are omni wheels, however other types of mobility devices are contemplated, such as pneumatic legs or hover systems using air or magnetic force or any combination thereof. Additionally, the exemplary embodiment uses four mobility devices 18a-18d, however any number of suitable mobility devices may be used. The mobility devices 18a-18d are configured to move the housing 13 and attached components from the docking station 100 to an area defined by a sanitation device user. The docking station 100 is configured to fit underneath the robot assembly 10.

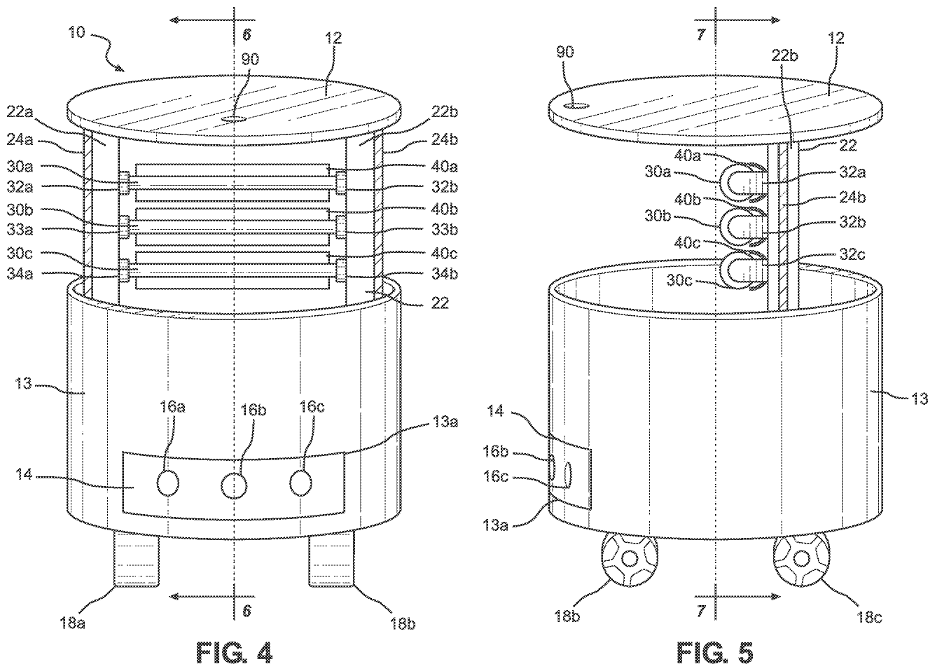

Referring to FIGS. 4 and 5 which show a front view and a right-side view of the robot assembly 10 in an extended position, respectively. In this position, the cover 12 is released from the housing 13. The robot assembly 10 includes a backing 22. The backing 22 is fixedly attached to cover 12. The backing 22 is rigid and may be composed of any suitable material such as a polymer or a metal that does not block wireless communication signals.

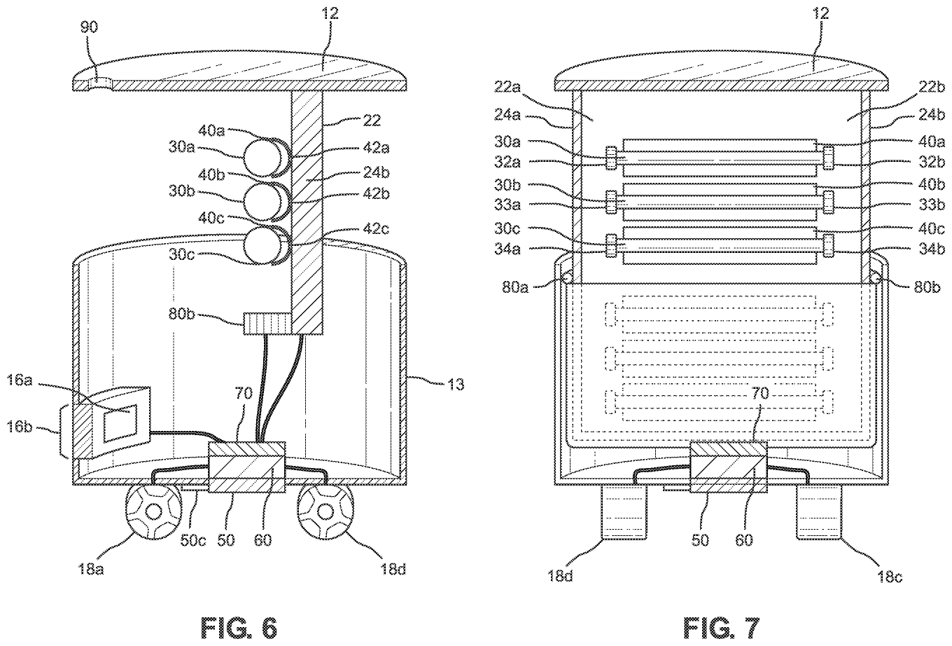

The robot assembly 10 includes at least one reflector 40a-40c; at least one light attachment 32a, 32b; 33a, 33b; 34a,34b; and at least one bulb 30a-30c. The at least one light attachment 32a, 32b; 33a, 33b; 34a,34b is attached to the backing 22. Each of the at least one reflector 40a-40c has a substantially curved shape. In the exemplary embodiment, each of the reflectors 40a-40c has a concave shape. In the exemplary embodiment, there are three reflectors 40a-40c, however any suitable number of reflectors may be used. Further, in the exemplary embodiment, each of the reflectors 40a-40c is a unitary piece. Each of the reflectors 40a-40c may be formed as a mirror, polished metal, metal plated polymer, or any other material that is suitable for reflecting light. It is contemplated that each of the reflectors may not be unitary and may be modular. It is envisioned that the reflectors can be formed as a concave MEMs mirror array, wherein the array is electrically connected to a power source 60 and control center 70 (shown in FIGS. 6 and 7) so that each of the mirrors in the array can be controlled independently.

In the exemplary embodiment, the bulbs 30a-30c are ultraviolet type C (“UV-C”) bulbs. However, any other suitable bulb capable of projecting anti-bacterial light is contemplated. Each of the curved reflectors 40a-40c has the cross-section that forms an arc with two end points. The at least one bulb 30a-30c is positioned at the midpoint, or approximate thereto, of the distance between the two endpoints of the arc formed by the corresponding curved reflector 40a-40c. Each bulb 30a-30c is configured to be secured to and electrically connected to a light attachment 32a, 32b; 33a, 33b; 34a,34b. Each of the light attachments 32a, 32b; 33a, 33b; 34a,34b is fixed to the backing 22. Each of the light attachments 32a, 32b; 33a, 33b; 34a,34b is electrically connected to the power source 60 and control system 70 (shown in FIGS. 6 and 7). In the exemplary embodiment, one bulb 30a-30c is used with one reflector 40a-40c. However, it is also contemplated that more than one bulb 30a-30c may be paired with a single reflector 40a-40c. Additionally, in this embodiment, two light attachments 32a, 32b; 33a, 33b; 34a, 34b are used with a single bulb 30a-30c. However, it is contemplated that any suitable number of light attachments 32a, 32b; 33a, 33b; 34a, 34b may be used with a single blub 30a-30c.

The at least one reflector 40a-40c; at least one light attachment 32a, 32b; 33a, 33b; 34a, 34b; and at least one bulb 30a-30c are arranged on the backing 22 in a manner such that when the robot assembly 10 is in the extended position, and at least one bulb 30a-30c is illuminated, light is reflected from the respective reflectors 40a-40c and directed away from the backing 22 of the robot assembly 10.

Furthermore, the at least one reflector 40a-40c; at least one light attachment 32a, 32b; 33a, 33b; 34a, 34b; and at least one bulb 30a-30c are arranged on the backing 22 in a manner such that each of the optical devices 16a-16c can capture information about whether light from the at least one bulb 30a-30c is projected onto a surface. For example, in operation, once a sector to be disinfected is selected by a user, the at least one optical device 16a-16c is configured to gather information to assess whether the at least one bulb 30a-30c was activated and how much light was projected onto the selected surface.

The robot assembly 10 includes at least one lift mechanism 24a, 24b. The at least one lift mechanism 24a is fixedly attached to a first side 22a of the backing 22 and a second lift mechanism 24b is attached to a second side 22b of the backing 22. In the exemplary embodiment, the backing 22 is formed as a rectangular solid having four vertical faces and two horizontal faces, wherein two of the vertical faces have a small area and two vertical faces have a large area. Each of the lift mechanisms 24a, 24b is placed on a vertical face with a small area on the backing 22. In an exemplary embodiment, there are two lift mechanisms 24a, 24b, wherein each lifting mechanism 24a, 24b includes a threaded rod.

Referring now to FIGS. 6 and 7, wherein FIG. 6 is a cross-section of the robot assembly 10 along plane B-B in FIG. 4 and FIG. 7 is a cross-section of the robot assembly along plane C-C in FIG. 5. The at least one lift mechanism 24a, 24b of the robot assembly 24a, 24b further includes at least one driver 80a, 80b. The drivers 80a, 80b are disposed inside the housing 13. In the exemplary embodiment, the robot assembly 10 includes two drivers 80a, 80b, wherein the drivers 80a, 80b are worm gears that are configured to drive the threaded rods. The at least one lift mechanism 24a, 24b is electrically connected to the power source 60 and communicates with control center 70. It is contemplated, however, that the at least one lift mechanism 24a, 24b may be any other suitable gear system, pneumatic system, actuator system, or a combination thereof that is capable of repeatedly separating and connecting the cover 12 and housing 13 of the robot assembly 10.

The robot assembly 10 includes at least one reflector mount 42a-42c. Each reflector mount 42a-42c is configured to attach a reflector 40a-40c to the backing 22. Each reflector mount 42a-42c may fix the attached reflector 40a-40c in a single orientation. However, it is also contemplated that each reflector mount 42a-42c may permit the attached reflector 40a-40c to rotate between different orientations. It is further contemplated is that at least one reflector mount 42a-42c is electrically connected to a power source 60 and control center 70.

As shown in FIG. 6, the at least one optical device 16a-16c and the at least one mobility device 18a-18d are electrically connected to the power source 60 and control center 70.

Referring to FIGS. 7 and 3, the robot assembly 10 includes a power source 60. In the exemplary embodiment, the power source 60 is a rechargeable battery. The power source 60 is configured to power all of the electrical components in the robot assembly 10. The power source 60 includes a charging interface 50. The charging interface 50 comprises the at least one connector prong 52a-52d. The at least one connector prong 52a-52d may be formed of gold, copper, or any other conductive material. The at least one connector prong 52a-52d is configured to receive the at least one connector pin 104a-104d. In the exemplary embodiment, each connector prong 52a-52d forms a V-shaped receiving pocket to receive the at least one connector pin 104a-104d therein. In operation, connecting the connector pins 104a-104d to the connector prongs 52a-52d enables an electrical connection between the outlet and the power source 60, thereby recharging the power source 60. However, it is contemplated that the docking station 100 may be configured as an inductive charging station not requiring the need for the physical contact between the connector pins 104a-104d and connector prongs 52a-52d.

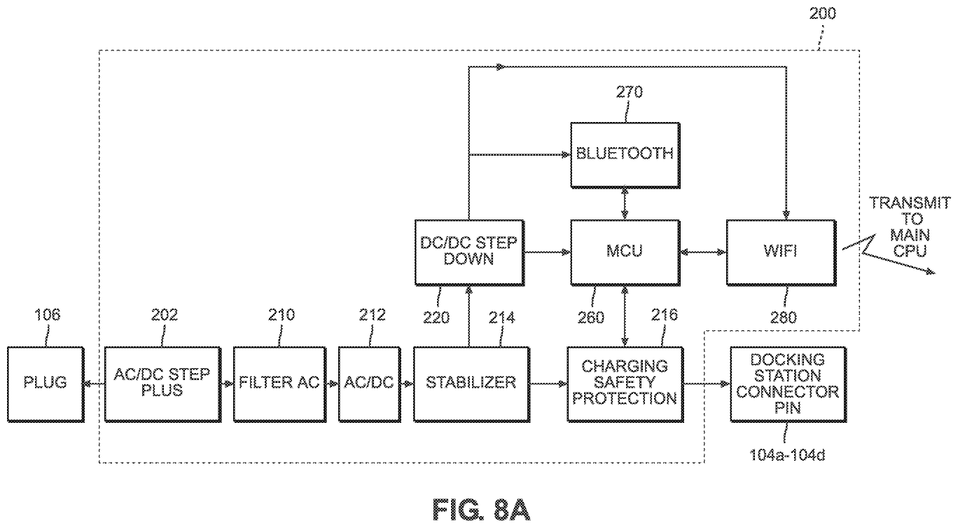

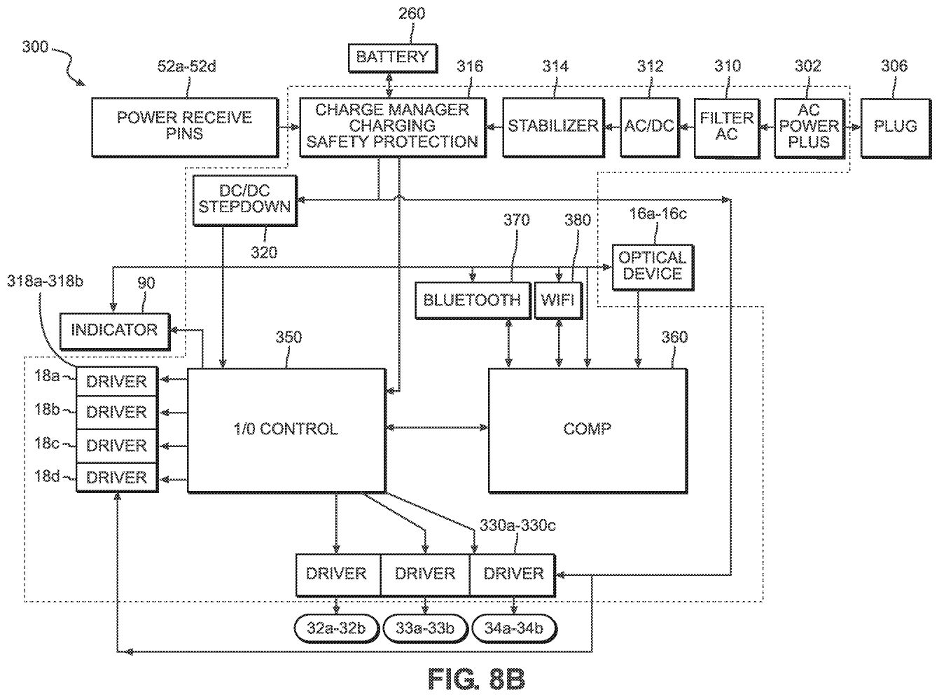

Now referring to FIG. 8A. The docking station 100 includes a docking station circuit (hereinafter “DSC”) 200. The DSC 200 is disposed inside of the casing 102 and is electrically connected to the plug 106 that is disposed outside of casing 102. The DSC 200 includes a docking station (hereinafter “DS”) AC power plus 202 to receive power once the plug 106 is connected to an outlet. The anticipated power source is AC power. A DS AC filter 210 is electrically connected to the DS AC power plus 202. The power output from the DS AC filter 210 is provided to a DS AC/DC converter 212. The power from the DS AC/DC converter 212 is provided to a DS stabilizer 214. The power from the DS stabilizer 214 is delivered to a DS DC/DC stepdown device 220 that is configured to step the voltage so that it is appropriate to power a MCU 260, DS Bluetooth 270, and DS Wi-Fi 280. The DS Bluetooth 270 and DS Wi-Fi 280 are configured to communicate with the MCU 260 as well as send and receive wireless communication signals. It is contemplated, however, that other suitable communication means may be used in place of the DS Bluetooth 270 and/or DS Wi-Fi 280 components or any combination thereof, wherein the suitable communication means is configured to emit and receive a wireless signal used in IoT devices.

The DS stabilizer 214 is electrically connected to a DS charge manager 216. The DS charge manager 216 is configured to operate as a charging safety protection to prevent overloading the DSC 200. The DS charge manager 216 is configured to communicate with the MCU 260. The DS charge manager 216 is configured to electrically connect with the at least one docking station connector pin 104a-104d.

Now referring the FIG. 8B. The robot assembly 10 includes a robot assembly circuit (hereinafter “RAC”) 300. The RAC 300 includes the control center 70, the at least one connector prong 52a-52d, the indicator 90, the at least one optical device 16a-16c, the power source 60, the at least one mobility device 18a-18d, the at least one light attachment 32a, 32b; 33a, 33b; 34a, 34b, and the plug 306. The control center 70 includes the same components as RAC 300 except the at least one connector prong 52a-52d, the indicator 90, the at least one optical device 16a-16c, the power source 60, the at least one mobility device 18a-18d, the at least one light attachment 32a, 32b; 33a, 33b; 34a, 34b, and the plug 306. The control center 70 is disposed inside the housing 13 of the robot assembly 10.

The RAC 300 includes a removable RA plug 306 that is configured to attach to a domestic power source, such as an outlet. The RAC 300 includes a robot assembly (hereinafter “RA”) AC power plus 302 to receive power once the RA plug 306 is connected to the outlet. The anticipated power source is AC power. An RA AC filter 310 is electrically connected to the RA AC power plus 302. The power output from the RA AC filter 310 is provided to an RA AC/DC converter 312. The power from the RA AC/DC converter 312 is provided to a RA stabilizer 314. The RA stabilizer 314 is electrically connected to a RA charge manager 316. The RA charge manager 316 is electrically connected to the at least one connector prong 51a-51d and power source 60. The RA charge manager 316 is configured to operate as a switch that can power the power source 60 via the at least one connector prong 51a-51d or via the plug 306. Additionally, the charge manager 316 is configured to operate as a charging safety protection to prevent overloading of the RAC 300 and the power source 60. As previously mentioned, in the exemplary embodiment, the power source 60 is a rechargeable battery.

The RA charge manager 316 is electrically connected to a RA DC/DC stepdown device 320 that is configured to step the voltage so that it is appropriate to power the indicator 90, an I/O Control 350, a RA Bluetooth 370, a RA Wi-Fi 380, a RA Comp 360, and the at least one optical device 16a-16c.

The RA charge manager 316 is electrically connected to at least one bulb driver 330a-330c. Each bulb driver 330a-330c is configured to operate as an interface between the RA charge manager 316 and the connected light attachment 32a, 32b; 33a, 33b; 34a, 34b. Each bulb driver 330a-330c is configured to regulate the power supplied to the connected light attachment 32a, 32b; 33a, 33b; 34a, 34b thereby controlling the intensity of lumens output from the at least one bulb 30a-30c.

The RA charge manager 316 is electrically connected to at least one mobility device driver 318a-318d. Each mobility device driver 318a-318c is configured to operate as an interface between the RA charge manager 316 and the at least one connected mobility device 18a-18d. The mobility device driver 318a-318d is configured to regulate the power supplied to the connected mobility device 18a-18d.

The RA DC/DC step down is electrically connected to indicator 90.

The RA DC/DC step down is electrically connected to the I/O Control 350. The I/O control 350 is configured to communicate with Comp 360 and control the peripheral devices including the indicator 90, the at least one bulb driver 330a-330c, and the at least one mobility device driver 318a-318d. Additionally, the I/O control 350 is configured to receive data from the RA charge manager 316. Data that the I/O control 350 may receive from the RA charge manager 316 is the capacity and charge amount in power source 60.

Comp 360 is capable of storing, executing, and/or processing data. In the exemplary embodiment, Comp 360 is computer. For example, Comp 360 may be configured to store a docking station ID for docking station 200. Comp 360 is configured to communicate with the I/O control 350 to cause the peripheral devices to operate in accordance with a stored program. The Comp 360 is configured to communicate with the RA Bluetooth 370 and RA Wi-Fi 380. The RA Bluetooth 370 and RA Wi-Fi 380 are configured to send and receive wireless communication signals. It is contemplated, however, that other suitable communication means may be used in place of the RA Bluetooth 370 and/or RA Wi-Fi 380 components or any combination thereof, wherein the suitable communication means is configured to emit and receive a wireless signal used in IoT devices. The RA Bluetooth 370 and/or RA Wi-Fi 380 is configured to communicate with the DS Bluetooth 270 and/or DS Wi-Fi 280.

Comp 360 is configured to communicate with the at least one optical device 16a-16c. The at least one optical device 16a-16c is capable of obtaining data and provide said data to Comp 360 for processing.

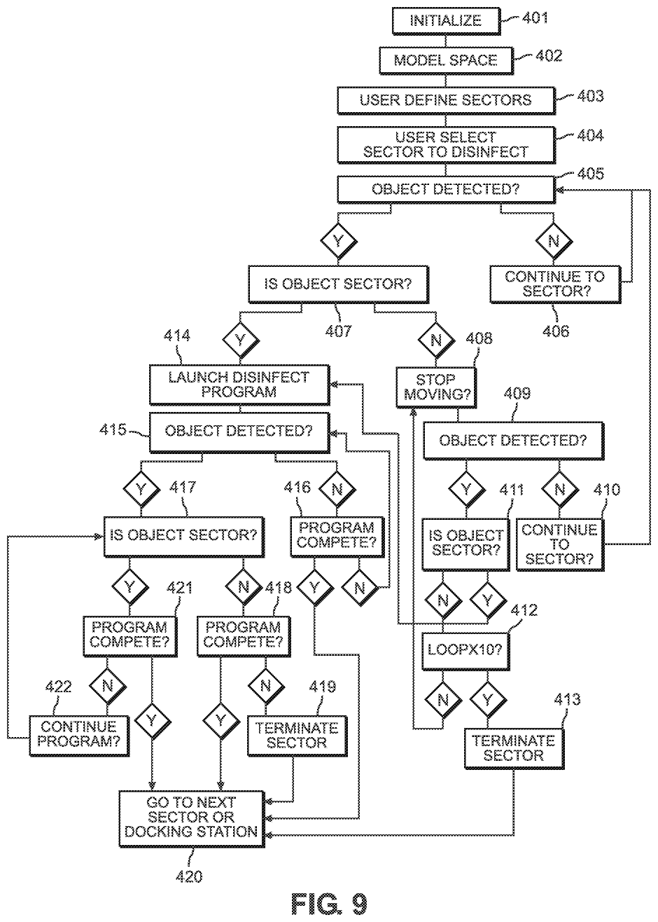

Now referring to FIG. 9, which is a flowchart 400 for sanitizing an area. The autonomous sanitation machine 1 including the robot assembly 10 and docking station 100 is configured to execute the sanitation method detailed in FIG. 9. In step 401, a user initializes the autonomous sanitation machine 1. The autonomous machine 1 will turn on and run a test to see if all auxiliary devices are connected and operable. Once step 401 is completed, the user can command the autonomous sanitation machine 1 to execute step 402, which is model a space. In this step, the autonomous sanitation machine 1 will use the auxiliary devices to capture an imprint of the space. For example, if the user executes step 402 in a one bedroom apartment, the autonomous sanitation machine 1 is configured to use the at least one mobility device 18a-18d and at least one optical device 16a-16c to capture data and model the one bedroom apartment. The captured and processed data will be stored in the memory of comp 360. It is further contemplated that the captured and processed data will be backed up in cloud-based storage that is accessible to the user. After the autonomous sanitation machine 1 has completed modeling the space in step 402, the user can proceed to step 403. In step 403, the user takes the modeled space and defines it into sectors, assigning sector values to sections of the modeled space. For example, in the modeled one bedroom apartment, the user can define the bedroom as sector 1, the kitchen as sector 2, the living room as sector 3, and so on. After the user confirms the sector values in the space, the user can execute step 404. In step 404, the user commands the autonomous sanitation machine 1 to sanitize a user defined sector. Is it contemplated that this command may be made on a mobile device with a connected app via Wi-Fi, Bluetooth, or another means of wireless communication, and/or on a computer that is capable of wireless communication. In the exemplary embodiment, the command may be made directly to autonomous sanitation machine 1. It is further contemplated that the command may be made through a cloud based online application. Once the user initiates step 404, the autonomous sanitation machine 1 will proceed to step 405.

In step 405, the autonomous sanitation machine 1 advances toward the sector selected by the user using the at least one mobility device 18a-18d. While the autonomous sanitation machine 1 is advancing toward the selected sector, the device will use the at least one optical device 16a-16c to look for foreign objects. This process will be done by comparing the data collected from the modeled space captured in step 402 and the newly captured space while travelling to the user selected sector. If an object is not detected, then the autonomous sanitation machine 1 continues to step 406, which is to proceed to the sector and look for an object. If an object is detected, then the autonomous sanitation machine 1 proceeds to step 407 and assesses whether or not the detected object is the selected sector. If the object is not the selected sector, the autonomous sanitation machine 1 moves to step 408 and stops moving toward the selected sector. For example, if an animal, person, or baby gate is detected as an object, then the autonomous sanitation machine 1 will stop moving. After the autonomous sanitation machine 1 stops moving in step 408, it proceeds to step 409 to see if the object is still detected. If the object is not detected anymore, then the autonomous sanitation machine 1 proceeds to step 410 and continues to the sector and returns to step 405.

If the object detected is still present, then the autonomous sanitation machine 1 proceeds to step 411 and determines whether the detected object is in the sector. If the object is not the sector, the autonomous sanitation machine 1 proceeds to step 412 and asks whether it has detected an object that is not the sector ten (10) times. If it has detected the non-sector object less than ten times, then the autonomous machine 1 returns to step 408. If the autonomous sanitation machine 1 has detected the non-sector object ten times, then it proceeds to step 413 and terminates the sanitation request. Thereafter, it goes to step 420 which is to travel to the next sector to be sanitized or return to the docking station 100 if there are no further sectors to sanitize. If a sanitization request is terminated without sanitizing the user selected sector, the autonomous sanitation machine 1 sends an alert to the user to notify them of the mission termination.

If the object detected by autonomous sanitation machine 1 in step 411 is in the sector, then the autonomous sanitation machine 1 proceeds to step 414 and launches a UV disinfection program. Once the UV disinfection program is launched, the robot assembly 10 transitions from the first, closed position (shown in FIG. 1) to the second, deployed position (shown in FIG. 4). The at least one bulb 30a-30c is illuminated to sanitize and disinfect the sector. During this process, the autonomous sanitation machine 1 proceeds to step 417 and uses the at least one optical device 16a-16c to see if an object is detected when the at least one bulb 40a-40c is illuminated. If no object is detected, the autonomous sanitation machine 1 proceeds to step 416 to see if the program is complete. If the program is not complete, the program continues while periodically checking to see if an object is detected. If the program is complete, the autonomous sanitation machine 1 proceeds to step 420 and travels to the next sector to be sanitized or returns to the docking station 100 if there are no further sectors to sanitize.

If an object is detected while the at least one bulb 40a-40c are illuminated and the UV disinfection program is being executed, then the autonomous sanitation machine 1 proceeds to step 417 and to see if the object is the sector. If the object is not the sector, then the autonomous sanitation machine 1 proceeds to step 418 and checks to see if the program is complete. If the program is not complete, then the autonomous sanitation machine 1 proceeds to step 419 and terminates disinfecting the section by turning off the at least one bulb 40a-40c. For example, in operation, if the at least one bulb uses a UV-C bulb, it can be harmful to humans and animals. If the autonomous sanitation machine 1 detects that an object has entered its field of vision, the bulbs will shut off, thereby preventing unnecessary harm to a potential human or animal. After the sector UV disinfection is terminated, the autonomous sanitation machine 1 will send an alert to the user to notify them that the disinfecting operation was interrupted. After the UV disinfection operation is terminated, the autonomous machine 1 proceeds to step 420 and travels to the next sector to be sanitized or returns to the docking station 100 if there are no further sectors to sanitize.

If at step 418, the autonomous sanitation machine 1 determines that the program was complete although an object was detected, the autonomous sanitation machine 1 will proceed to step 420 and travel to the next sector to be sanitized or return to the docking station 100 if there are no further sectors to sanitize.

If at step 417 the autonomous sanitation machine 1 determines that the object detected is in the sector, then it will proceed to step 421 and check to see if the program is complete. If the disinfecting program cycle is complete then the autonomous sanitation machine 1 will proceed to step 420 and travel to the next sector to be sanitized or return to the docking station 100 if there are no further sectors to sanitize.

If at step 421 the autonomous sanitation machine 1 determines that the program is not complete, it will proceed to step 422 and continue running the program and will proceed to step 417 to periodically check to find an object.

The flowchart in FIG. 9 is exemplary and not exhaustive. It is further contemplated that the user can terminate, pause, or otherwise interrupt the program on demand. It is also contemplated that the autonomous sanitation machine 1 will begin at step 404 once a user selects a new sector to be sanitized.

Throughout the description, including the claims, the term “comprising a” should be understood as being synonymous with “comprising at least one” unless otherwise stated. In addition, any range set forth in the description, including the claims should be understood as including its end value(s) unless otherwise stated. Specific values for described elements should be understood to be within accepted manufacturing or industry tolerances known to one of skill in the art, and any use of the terms “substantially” and/or “approximately” and/or “generally” should be understood to mean falling within such accepted tolerances.

Although the present disclosure herein has been described with reference to particular embodiments, it is to be understood that these embodiments are merely illustrative of the principles and applications of the present disclosure.

It is intended that the specification and examples be considered as exemplary only, with a true scope of the disclosure being indicated by the following claims.

1. An autonomous sanitation machine comprising:
a robot assembly and docking station;
wherein the robot assembly includes at least one mobility device, at least one optical device, a Comp, and at least one bulb;
wherein the at least one optical device is configured to capture data from a space;
wherein the Comp is configured to process the captured data and model it as an imprint;
wherein the user assigns a sector within said imprint; and
wherein the at least one bulb is configured to sanitize the user assigned sector.

2. The autonomous sanitation machine of claim 1, wherein the at least one optical device is a 3D camera.

3. The autonomous sanitation machine of claim 1, wherein the at least one bulb is configured to emit UV-C light.

4. The autonomous sanitation machine of claim 1, wherein the robot assembly is configured to move between a first extended position and a second closed position.

5. The autonomous sanitation machine of claim 4, wherein the at least one bulb is exposed when the robot assembly is in the first extended position and the at least one bulb is inside of the robot assembly when the robot assembly is in the second closed position.

6. The autonomous sanitation machine of claim 1, wherein the robot assembly includes at least one reflector and at least one reflector mount.

7. The autonomous sanitation machine of claim 6, wherein the at least one reflector is substantially curved.

8. The autonomous sanitation machine of claim 6, wherein the at least one reflector is attached to the at least one reflector mount; wherein the at least one reflector is configured to rotate relative to the at least one reflector mount.

9. A method for sanitizing an area with an autonomous sanitation machine comprising:
using an autonomous sanitation machine to model a space;
defining sectors in the space by a user;
selecting the sector to be sanitized;
launching a sanitization program causing the autonomous sanitation machine to go to the selected sector; and
disinfecting the selected sector.

10. The method of claim 9, including searching for an object by the autonomous sanitation machine, detecting an object, determining if the object is the sector, and terminating the sanitization program if the object is not the sector.

11. The method of claim 10, including alerting the user of the terminated program if the sanitization program is terminated.

12. The method of claim 10, including stopping the at least one mobility device of the autonomous sanitation machine when the object is detected.

13. The method of claim 10, including turning off the at least one bulb of the autonomous sanitation machine when the object is detected.
